(19) United States
(12) Patent Application Publication
(10) Pub. No.: US 2022/0229046 A1
(43) Pub. Date: Jul. 21, 2022

(54) COLLECTION CONTAINER

(71) Applicant: EnteroBiotix Limited, Aberdeen (GB)

(72) Inventors: James MCILROY, Aberdeen (GB); Gregor RUSSELL, Aberdeen (GB); Vlad MASALSKI, Aberdeen (GB); Helen STRANGE, Aberdeen (GB); Patricia DEL RIO, Aberdeen (GB); James CLARK, Aberdeen (GB); Nicolas ROBINSON, Aberdeen (GB); Gregor ANDERSON, Aberdeen (GB)

(73) Assignee: EnteroBiotix Limited, Aberdeen (GB)

(21) Appl. No.: 17/609,614

(22) PCT Filed: May 11, 2020
(86) PCT No.: PCT/GB2020/051138

§ 371 Date: Nov. 8, 2021

(30) Foreign Application Priority Data
May 8, 2019 (GB) 1906501.0

(51) Int. Cl. G01N 33/52; A61B 10/00; A47K 11/04

(52) U.S. Cl. CPC G01N 33/528; A47K 11/04; A61B 10/0038

(57) ABSTRACT

The invention relates to a flexible specimen collection container for use with a toilet frame comprising: a sealable opening for receiving stool directly from a donor; a first compartment for receiving and holding stool, said first compartment coupled to said opening; a second compartment for collecting and holding fluid received from the donor through the sealable opening, the second compartment being in fluid communication with said first compartment and configured to substantially separate the fluid from the stool. At least one port may be connected to said first compartment and being configured to pass at least a portion of the stool.

The invention relates to a specimen collection container for the collection and processing of faeces and derivatives thereof, for example, an intestinal microbiome medicinal product (IMMP), a toilet frame and a system for the collection and processing of faeces, and derivatives thereof, for example intestinal microbiome material comprising a specimen collection container and a toilet frame.

### BACKGROUND

The field of intestinal microbiome transplantation, also known as faecal microbiota transplantation (FMT), is much studied and there has been rapidly growing medical, scientific and public interest in the treatment since it was discovered that the gastrointestinal tract and the organisms therein have a significant impact on the mood, appetite and health of the host, be it person or animal. Treatments for ailments associated with imbalances of microbial communities within the intestinal tract, such as recurrent Clostridium (recently reclassified as Clostroides) difficile infection have historically been lacking, but with FMT cure rates for such infections, particularly those that are recurrent in nature, have been reported as high as 90%. There is also a growing body of evidence suggesting that this treatment and procedure can be beneficial for a number of intestinal and extra intestinal disease, such as obesity, irritable bowel syndrome and metabolic disease.

FMT is a multi-stage process which involves donation of stool from a healthy donor, preparation of the donation and delivery into a patient. Each of these processes has various nuances and variations depending on the disease being targeted and other considerations such as patient demographic and cost.

More refined and standardised manufacturing processes, such as those implemented to manufacture intestinal microbiome medicinal products, are also multi-stage and involve a number of manipulations.

One factor that is always a concern for any manufacturer of products for use in FMT or an entity developing and manufacturing intestinal microbiome medicinal products is that often the donation can be diluted and degraded prior to processing because of contact with pollutants, in particular urine. Separating urine out of the stool sample is important for increasing the quality of the processed product. Collecting donation from a rigorously screened donor within a controlled environment can be expensive (requiring a trained professional) and take time to procure, therefore each donation that is lost or partially degraded can represents a significant loss. Prior art collection bags merely rely on the donor to avoid mixing urine with their donation, this involves the trained professional spending time explaining how the donation should work, which can cause embarrassment to the donor.

Additionally, donations that are not of high quality may be more liquid in nature as defined by scales such as the Bristol Stool Scale and are therefore not appropriate for processing and downstream drug substance and drug product manufacture. Prior art collection bags do not enable the assessment of this critical quality attribute without opening the device and breaking the closed system.

Human donors often find the donation process unpleasant and as such donors are relatively hard to find. Advances to technology in this area which improves the chances of a successful (non-polluted) donation whilst making the process easier and less unpleasant for the donor are of vital importance.

Beyond collecting donated faecal matter that is subsequentially used as material for contemporary FMT or as starting material for a more refined intestinal microbiome medicinal product, collection devices are also used to collect donations that are then analysed for research or as part of clinical trials. Prior art collection bags do not enable an operator or a donor to remove a standardised amount of the donation in an aseptic and convenient manner without opening the device. Current state of the art mandates that a donor must use a scooping instrument to remove aspects of the donation from a receptacle.

Manufactures of intestinal microbiome medicinal products and those who collect material that is intended for human application as part of contemporary FMT may wish to collect donations from home or from a partially and sometimes substantially uncontrolled environment. In this situation, establishing adequate chain of custody is vital. Those skilled in the art will appreciate that one method of establishing chain of custody is through linking the microbial profile of a particular donation to a previously profiled sample.

Prior art bags do not able the removal of a standardised portion of a donation suitable for microbial profiling as part of a method to establish chain of custody prior to subsequent downs stream processing.

### BRIEF SUMMARY OF THE DISCLOSURE

In accordance with the present inventions there is provided a specimen collection container, a toilet frame and a system for the collection and processing of faeces comprising a specimen collection container and a toilet frame according to the appended claims.

A first aspect of the present invention comprises a specimen collection container for the collection and processing of faeces and configured for use with a toilet frame, the container comprising: a sealable opening for receiving stool directly from a donor; a first compartment for receiving and holding stool, said first compartment coupled to said sealable opening; and one or more further compartment:—
a. a further compartment being connected to the first compartment via a sealable port for transferring stool, and/or
b. a further compartment being for collecting and holding fluid received from the donor through the sealable opening, wherein the further compartment is in fluid communication with said first compartment and configured to substantially separate the fluid from the stool

The compartment for transferring stool and the compartment for collecting and holding fluid may be separate compartments. The specimen collection container may consist or comprise of a first compartment and a compartment for transferring stool. The specimen collection container may consist or comprise of a first compartment and a compartment for collecting and holding liquid. The specimen collection container may consist or comprise of a first compartment, a compartment for transferring stool and a compartment for collecting and holding liquid.

The specimen collection container may therefore comprise: a sealable opening for receiving stool directly from a donor; a first compartment for receiving and holding stool, said first compartment coupled to said sealable opening; a second compartment for collecting and holding fluid received from the donor through the sealable opening, the second compartment being in fluid communication with said first compartment and configured to substantially separate the fluid from the stool. The fluid may be urine, the fluid may be liquified derivatives of stool after processing, or a combination thereof.

At least one port may be connected to said first compartment and being configured to pass at least a portion of the stool. The port may be sealable. The port may be connecting the first compartment to a further compartment, or connecting the first compartments to the external environment. Consequently, faecal matter held within first compartment may be transferred through the port from first compartment to a further compartment, or outside of the container.

In certain embodiments, the at least one port connected to said first compartment is configured to pass at least a portion of the stool which may be a diluted stool sample comprising one or more of: saline, glycerol, trehalose, sucrose, cysteine, short chain fatty acids, or other added material.

In certain embodiments the specimen is faecal matter. The faecal matter for collection may be unprocessed stool delivered directly by the donor. When the faecal material is to be used in FMT or as a IMMP, stools provided preferably conform to Type 3 and/or 4 on the Bristol Stool Scale

In other embodiments, the specimen is intestinal microbiome material. For example, a product derived from faecal matter, optionally processed faecal matter.

By providing a specimen collection container which comprises two compartments, the second being in fluid communication with the first, the process of donating faeces for FMT or for the manufacture of intestinal microbiome medicinal products is made simpler and more efficacious by means of separating urine and other undesirable liquids from the donated stool which is required transplantation. The compartment for collecting and holding fluid may be provided below the first compartment, when in use, such that fluid flow under gravity from the first to the second compartment.

Another advantage of the present invention is that when donors are patients with suspected gastrointestinal tract infections, the process of collecting faeces and processing material prior to testing is made simpler and safer by reducing the risk of operator contact with the material.

In an embodiment of the present invention, the first compartment and the compartment for collecting and holding fluid are separated by a filter which allows fluid ingress to the compartment for collecting and holding fluid from the first compartment.

The use of a filter which allows fluid ingress into the compartment for collecting and holding fluid from the first compartment will further aid in the removal of undesirable liquids such as urine from the stool.

The use of a filter and the compartment for collecting and holding fluid also allows an operator to assess if a donation is not of appropriate consistency to be rendered suitable for processing, particularly for processing for the purposes of FMT or as an IMMP. For example, stool samples conforming to Type 3 and/or 4 on the Bristol Stool Scale would be retained on the first compartment side of the filter. Only urine being provided in the compartment for collecting and holding fluid side of the filter would therefore be indicative of a good stool sample. An inappropriate sample for use in FMT or as an IMMP would result in some of the donated material being present in the compartment for collecting and holding fluid; a fact confirmed easily be visual assessment (e.g those conforming to type 6 or 7 of the Bristol Stool Scale).

In an embodiment of the present invention the filter is a constriction in the material of the container between the first compartment and the compartment for collecting and holding fluid. For example, a constriction that permits fluid to pass through but not healthy solid faecal material.

Using a constriction as the filter is a cost-effective way of removing said undesirable liquids because no further materials or elements are required.

In certain embodiments the filter comprises a porous membrane. More specifically, the porous membrane may be one of: a perforated polymer sheet, a mesh or the like. It will be understood that when the porous membrane is a mesh, the mesh pore size is such that fluid may pass through the mesh but solid and particulate material will not. For example, the mesh pore size may be more than 0.1 μm. The mesh pore size may be less than 300 μm. The mesh pore size may therefore be from about 0.1 to about 300 μm.

In certain embodiments, there may be more than one filter.

In certain embodiments the specimen collection container is tapered from the first compartment to the compartment for collecting and holding fluid. Tapering the collection container will allow it to fit easily into a large number of toilet bowls, thus improving the hygiene as it will not touch the sides of the toilet bowl or the water in the toilet bowl.

In certain embodiments the compartment for collecting and holding fluid may be a sump. Additionally, or alternatively, the specimen collection container comprises a compartment for collecting and holding fluid and a sump, the sump being in fluid communication with said compartment for collecting and holding fluid. More specifically, the sump is a fluid sump. The invention may consist of any combination, or all, of the compartments described herein and a sump. When a sump is provided in fluid communication with said compartment for collecting and holding fluid, the compartment still has capacity to collect and hold fluid should the sump overflow, or the fluid communication is closed.

Including a sump (for example, in fluid communication with said compartment for collecting and holding fluid) allows undesirable liquid to be transferred, under gravity, to a portion of the container from which it is unlikely to remix with the donated stool whilst the container is in transit. The sump may therefore be provided under the compartment for collecting and holding fluid when orientated for use.

An added benefit of the aforementioned tapered element and the sump is that a donation that is less viscous will naturally move down the container into the sump. In this sense, the constriction and the sump act as features that help an operator to assess the suitability of the donation for downstream manufacturing and patient use.

In certain embodiments, the sump is in fluid communication with said compartment for collecting and holding fluid (or first compartment when the compartment for collecting and holding fluid is a sump) via a one-way valve, a drain hole or an orifice.

In certain embodiments an absorbent material is located in said sump. In this way, the separation of the stool sample and the fluid in the container can be further enhanced by locking the fluid into the absorbent material.

In certain embodiments the specimen collection container comprises a device for detecting and indicating the pH of the fluid. More specifically, the device for detecting and indicating the pH of the fluid is located in said sump.

In certain embodiments the compartment for collecting and holding fluid (or first compartment when the compartment for collecting and holding fluid is a sump) is fluidly isolated from said sump by a seal. In this way, a barrier between the stool sample and the fluid in the sump is provided preventing remixing of the stool and the fluid in the sump.

In certain embodiments the seal is at least one of the following; a welding line comprising material for welding, a clamp and clamping line comprising material suitable for clamping, an adhesive, magnetic fasteners, a hook and loop fastener, a zip-lock, or a snap fit seal.

In certain embodiments a destructible portion is located between said first compartment and sump to allow removal of said sump from said first compartment, optionally between the compartment for collecting and holding fluid and the first compartment (e.g when the compartment for collecting and holding fluid is a sump), or between the sump and the compartment for collecting and holding fluid. For example, a weakened line maybe provided in the seal separating compartments, or sump form compartments.

Removing the sump after sealing will decrease the size of the collection container making it easier to transport and minimises the chance of including undesirable liquids with the stool. It will additionally make processing the sample within the specimen collection container easier by eliminating any residual chance that urine maybe inadvertently pushed back into the main body of the container during the processing steps.

In certain embodiments, the sump contains filtered material that can be used for subsequent downstream processing and analysis.

In certain embodiments, the first compartment comprises a filter configured to separate the first compartment into two sub-compartments. Whilst the first compartment may have a single filter, an alternative is for more than one filter to be provided and configured to separate the first compartment into more than two sub-compartments. The filter or filters may be arranged longitudinally within the first compartment when the container is in the orientation for use. Such a structure enables processing of the stool as it is manipulated into transverse movement across the horizontal axis of the first compartment. The filter or filters may instead be arranged horizontally within the first compartment when the container is in the orientation for use. Such a structure enables processing of the stool as it is manipulated into perpendicular movement down the vertical axis of the first compartment. The first compartment may include both longitudinally and horizontally aligned filters in order that manipulation in any direction will process stool within the first compartment. The filters may be provided in sequential grades of filter. When multiple filters are provided longitudinally with in the first compartment, the filters graduate by the maximum size of object allowed to pass therethrough, the filter permitting the largest object to pass being provided at the top of the first compartment when orientated for use (i.e sequential grades of filters are provided). When multiple filters are provided horizontally with in the first compartment, the filters graduate by the maximum size of object allowed to pass therethrough, the filter permitting the largest object to pass being provided at one side of the first compartment when orientated for use (ie sequential grades of filters are provided). More specifically, the filter comprises a porous membrane. More specifically, the porous membrane may be one of: a perforated polymer sheet, a mesh or the like. It will be understood that when the porous membrane is a mesh, the mesh pore size is such that small particulates and microbiome from the stool sample and also fluids can pass through the mesh but roughage solids from the stool sample will not. The small particulates and microbiome from the stool sample and also fluids (such as saline and glycerol added to the sample) are the elements used in the FMT process.

In certain embodiments the mesh size of the porous membrane separating the first compartment into two or more sub-compartments is from about 0.1 to about 300 μm. Optionally 200 microns or 300 microns. More specifically the mesh size may be 280 microns. The first filter may have a mesh size of from 300 to 200 microns (about 280 microns). The last filter in the sequence may have a mesh size of less than 50 microns, about 5 to 0.1 microns or about 0.1 micron.

In certain embodiments the specimen collection container is formed of a material suitable for visual inspection of the contents. More specifically the material may be transparent.

Visual inspection of the contents may allow the donation to be rejected or accepted in a “first pass” test and allow a user to manipulate the stool into the various inlets, outlets and compartments.

In certain embodiments the specimen collection container is formed of a deformable material suitable for manipulation of the contents by hand. More specifically, the collection container is formed of a flexible material. In certain embodiments the flexible material is formed of a flexible polymer. Yet more specifically, the flexible material may be polyethylene, polyvinyl chloride (PVC), ethylene vinyl acetate (EVA) or the like.

In certain embodiments the specimen collection container is formed of a non-toxic, non-pyrogenic material.

In certain embodiments the specimen collection container is formed of a material which can be sterilised in order to sterilise the container, for example a material that can be irradiated and/or treated chemically (e.g washing with ethylene oxide).

Forming the collection container out of a deformable material will allow a user to manipulate the stool into the various inlets, outlets and compartments.

As described, the specimen collection container may include a further compartment connected to the first compartment via a sealable port for transferring stool. This enables a portion of the deposited stool to be manipulated via the port into a separate compartment to the substantial part of the stool. When carried out prior to processing of the stool, this enables a portion of unprocessed stool to be retained, whilst processing of most of the specimen is processed.

In certain embodiments the compartment for transferring stool is detachable from said collection container.

Including a detachable compartment for transferring stool allows a user to urge some of the sample into the third compartment from the first compartment that can then be removed for testing without needing to delay the transportation of the rest of the donation located in the first compartment.

In certain embodiments the sealable port is sealable by at least one of the following; welding section comprising material for welding including, not limited to, a clamp and clamping line comprising material suitable for clamping, an adhesive, magnetic fasteners, a hook and loop fastener, a zip-lock, or a snap fit seal.

There are many alternative means of sealing such a port, the above is not an exhaustive list of all possible means.

In certain embodiments the internal diameter of the sealable port is up to 10 mm. More specifically, the internal diameter of the sealable port is between 2 mm and 10 mm. Preferably, the internal diameter of the sealable port is 5 mm.

In certain embodiments a destructible portion is located between said compartment for transferring stool and said first compartment. More specifically, the destructible portion may be located between said first compartment and the compartment for transferring stool to allow removal of compartment for transferring stool from said first compartment. For example, a weakened line maybe provided in the seal separating the first and compartment for transferring stool, e.g a perforated line.

Including a destructible portion means that the compartment for transferring stool, or sump, can be removed easily without requiring tools. Alternatively, specialist tools could be used on the destructible portion to make the removal easier and cleaner, for example scissors.

In certain embodiments the compartment for transferring stool further comprises at least one sealable external port.

An external port can be used to emit samples for testing, by urging or by gravity, the port can be sealed for transportation and to prevent leaks.

In certain embodiments the sealable opening is held closed by a deformable material clamp circumscribing the sealable opening until a force is applied to deform said deformable material clamp moving same to an open position.

Having the sealable opening held closed until a force is applied means that odour and moisture from the collection container will cease as soon as it is removed from the frame because the force holding it open will cease. This will also prevent losses and mess due to the donor or trained professional dropping the container.

In certain embodiments the specimen collection container comprises an indicated clamp area which can be closed by a clamp for closing said sealable opening.

Using a clamp on the sealable opening will prevent odour and moisture from leaving the bag after a donation has been made and mean that the container can be sealed for transportation or storage. This will also prevent losses and mess due to the donor or trained professional dropping the container.

In certain embodiments, the first compartment is separated into sub-compartments below the clamp area.

In certain embodiments the sealable opening provides unobstructed access to the first compartment. More specifically, the sealable opening provides the widest diameter of the specimen collection container. In this way, defecation by a donor into the collection container is as simple and natural as possible. By having the sealable opening across the full width of the collection container opening, specimen collection is made easier.

In certain embodiments, the first compartment comprises one or more inlet ports. More specifically, the inlet ports may be unidirectional inlet ports. In this way, fluids containing cryoprotectants, lyoprotectants, reducing agents and small molecules, including, but not limited to, glycerol, trehalose, sucrose, cysteine, short chain fatty acids and/or saline can be introduced into the first compartment during the processing of the specimen in the container Inlet ports can be arranged as opening onto the stool donation itself or, when the first compartment is separated into sub-compartments, the one or more inlet ports open into the sub-compartment which will contain the small particulates and microbiome from the stool sample and also fluids once the deposited stool specimen has been passed through the porous filter between the sub-compartments of the first compartment. For example, the one or more inlet ports open in the sub-compartment at the top of the first compartment, when the filter or filters are arranged horizontally within the first compartment when the container is in the orientation for use. Optionally, when filters are arranged longitudinally within the first compartment, the one or more inlet ports open in the sub-compartment defined by the filter permitting the largest maximum size of object to pass therethrough and the side of the compartment itself.

In certain embodiments, the first compartment comprises one or more outlet ports. More specifically, the outlet ports may be unidirectional ports. In this way, samples may be removed from the container for sampling purposes. When the first compartment is separated into sub-compartments, the one or more outlet ports maybe provided in the opposite positions as described with respect to the inlet ports.

A further embodiment of the present invention comprises at least one attachment element for attaching the container to a toilet support frame.

Including an attachment element for use with a complimentary element on the toilet support frame will allow the collection container to be quickly and simply attached to the toilet support frame.

In certain embodiments the at least one attachment element comprises an aperture in the material of the container wherein the aperture is configured to engage with a clip element on a support frame.

An aperture and clip-based connection element will allow a user to attach the collection container with ease because the aperture and the clip will be recognisable both visually and by feel.

In certain embodiments the container comprises at least one handle.

Including a handle as part of the collection container means that a user can carry the collection container in the correct orientation without needing to come into contact with the donated stool through the packaging, which some may find unpleasant.

In certain embodiments the specimen collection container comprises a timing mechanism.

It is important to know how much time has elapsed between donation and use as the stool may degrade over time. Six hours is an important timescale for stool donations.

In certain embodiments, the timing mechanism is a stopwatch.

In certain embodiments the stopwatch is configured to start recording time when the sealable opening is sealed.

In certain embodiments the timing mechanism comprises one or more of: a time bar; a timer configured to change colour over a defined time period; and a timer configured to provide a visual indicator due to a chemical reaction based on time elapsed.

In certain embodiments the specimen collection container is foldable. More specifically, the specimen collection container is foldable about a transverse fold line in order to shorten its longitudinal dimension. In this way, the specimen collection container can be folded to keep it above the water level in the toilet bowl.

In certain embodiments, the specimen collection container is retained in its folded configuration by a retaining element (e.g. hook and loop, adhesive).

In certain embodiments, the specimen collection container has an internal container volume of up to 2.5 litres. More specifically, the combined internal volume of the first and second compartments is between 1.5 litres to 2.5 litres.

In certain embodiments, the specimen collection container is used in human specimen collection.

In certain embodiments, the specimen collection container is used in non-human animal specimen collection.

A further aspect of the present invention comprises a toilet support frame for use with a specimen collection container, the frame comprising an opening through which a donor may deposit a stool sample; at least one clip for engaging an attachment element of a specimen collection container, and a urine shield configured to deflect urine away from said opening.

The urine shield allows the donor to avoid accidentally urinating into or onto the collection container by being configured to ensure that when the anus of the donor is over the collection container, their urethra is on the “safe” side of the urine shield, meaning that no urine will flow into or towards the container. The urine shield may be configured to be unisex.

In certain embodiments the urine shield comprises a deformable flap configured to be moved from a stowed position to a deployed position in which it is operable to deflect urine away from the opening.

Several features of the present invention can be placed into a stowed position such that they can be transported or stored whilst taking up a minimal amount of space. More specifically, in certain embodiments, the toilet support frame is initially provided in a flat, stowed configuration.

In certain embodiments a location tab is operable to engage with a toilet to secure the toilet support frame in the correct orientation and position relative to the toilet bowl.

The location tab facilitates correct fitting of the toilet frame and allows a user to place the frame in the correct position and orientation by making the location tab abut the rear of the toilet rim, ensuring that when the collection container is connected it is always in the optimal position.

In certain embodiments the toilet support frame comprises a plurality of support arms for positioning under a toilet seat or directly on a toilet bowl.

Placing support arms under the toilet seat may prevent the frame from slipping and moving around as a donor gets into position above the collection container.

In certain embodiments the support arms are attached to the frame by a hinge.

Putting the support arms on a hinge allows them to be stowed or deployed according to the dimensions of the toilet or stowed during transportation or storage such that the frame takes up less space.

In certain embodiments the hinge is a living hinge.

Using a living hinge means that the hinge can be formed integrally with the frame and from the same material, reducing manufacturing costs compared to adding a further component.

In certain embodiments the toilet support frame is a unitary construction.

Forming the frame as a unitary construction may be cheaper and will mean that there are no weak points in the frame such as adhered parts.

In certain embodiments the frame has a fluid resistant surface.

Making the frame fluid resistant will ensure that if it comes into contact with liquid it will not bend or buckle due to losing integrity or taking on excess mass from the liquid.

In certain embodiments the frame comprises a removable and replaceable lid configured to close the opening through which a donor may deposit a stool sample.

In certain embodiments, the specimen collection container is a container according to an aspect of the present invention.

An aspect of the present invention comprises a system for the collection and processing of faeces comprising a specimen collection container and a toilet support frame.

In certain embodiments, the specimen collection container is a container according to an aspect of the present invention and the toilet support frame is according to an aspect of the invention.

### BRIEF DESCRIPTION OF THE DRAWINGS

Embodiments of the invention are further described hereinafter with reference to the accompanying drawings, in which:

FIG. 1 shows an aspect of the present invention comprising a specimen collection container which comprises first, second and third compartments.

FIG. 2 shows an embodiment of the present invention comprising a first, second and third compartment, further comprising a sump.

FIG. 3 shows a further aspect of the present invention comprising a toilet frame, with a lid.

FIG. 4A shows a further embodiment of the toilet frame comprising support arms on hinges.

FIG. 4B shows a further embodiment of the toilet frame in place on a toilet rim.

FIG. 5A shows a further aspect of the present invention, a system for faecal matter collection and processing, comprising a toilet frame and a disengaged complimentary specimen collection container.

FIG. 5B shows a further embodiment of the system for faecal matter collection and processing comprising a toilet frame and an engaged complimentary specimen collection container.

### DETAILED DESCRIPTION

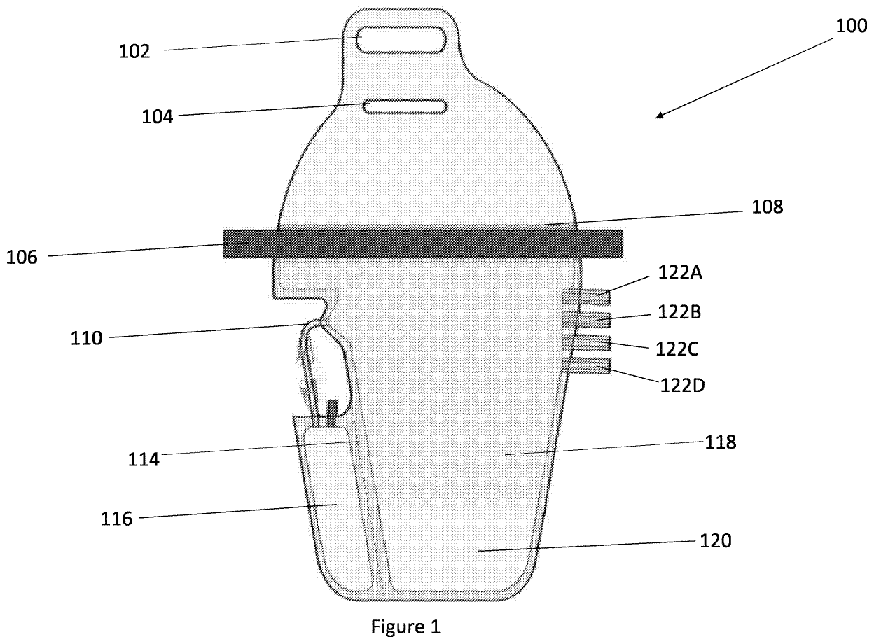

FIG. 1 shows a specimen collection container 100 formed of a transparent flexible polymer material which comprises first compartment 118 a second compartment 120 and a third compartment 116. In this embodiment the first compartment 118 comprises a mesh such that the first and second compartments are in fluid communication and fluid is separated from the specimen deposited in the first compartment 118. Non-fluid matter will not enter the second compartment 120 without urging. The first compartment may be separated into two sub-compartments by a porous membrane or mesh. The holes in the mesh may be between 200 and 300 microns in size. The porous membrane (not shown) extends longitudinally (parallel to the side walls of the container) from the clamp locator 108 towards the second compartment 120. The porous membrane is able to separate the stool sample into a roughage containing sample of larger particulates and a filtered stool sample comprising small particulates and microbiome from the stool sample and also fluids once the deposited stool specimen. The filtered stool sample may then have additives such as glycerol, trehalose, sucrose and/or saline added before the sample is processed further into a final product.

The specimen collection container further comprises a handle 102 and a clip aperture 104. The handle 102 is located distant from the stool sample deposited during a donation and can therefore be used to transport the specimen collection container after donation with a lowered risk of contamination to the stool sample. The clip aperture 104 is a means of connecting the specimen collection container to a toilet frame. It should be understood that any suitable connection means may be used, for example, adhesive, a snap on connector, hook and loop tape, poppers, buttons, fast release mechanism, zip tie, VELCRO® or magnets.

The specimen collection container 100 further comprises a clamp locator 108, being a line drawn on the material of the container 100. In other embodiments (not shown), the clamp locator 108 is in the form of one or more of: written instructions, a coloured area, a dotted line, tactile features such as bumps or the like. A clamp 106 is applied around the specimen collection container in a position indicated by the clamp locator 108. Although not shown in the depicted embodiment, after a stool sample deposit has been made into the collection container 100 and when the clamp 106 has been secured around the container, the portion of the specimen collection container comprising the clip aperture 104 and the handle 102 can be removed as superfluous. This has the advantage that the specimen collection container cannot then be reused. A destructible portion may be provided in the collection container 100 at a position above the clamp locator 108, on the same side of the clamp as the handle 102.

In the depicted embodiment of FIG. 1, a third compartment 116 is connected to the first compartment 118 and the second compartment 120 by a destructible portion being line of weakening 114 and a port 110 through which a stool sample can be urged. The port is 5 mm in diameter and can be sealed by a tool known in the art. This detachable third portion 116 can be filled with a sample, labelled and kept for destructive analysis whilst the remaining sample in the first compartment is prepared for use.

In this embodiment four further fluid ports 122A-D are shown connected to the first compartment, these can be used to admit fluids such as saline and glycerol or to emit further samples or prepared donations into external containers or a recipient. The fluids ports comprise unidirectional valves to ensure the contents of the first compartment remain sterile once the container 100 is sealed by clamp 108.

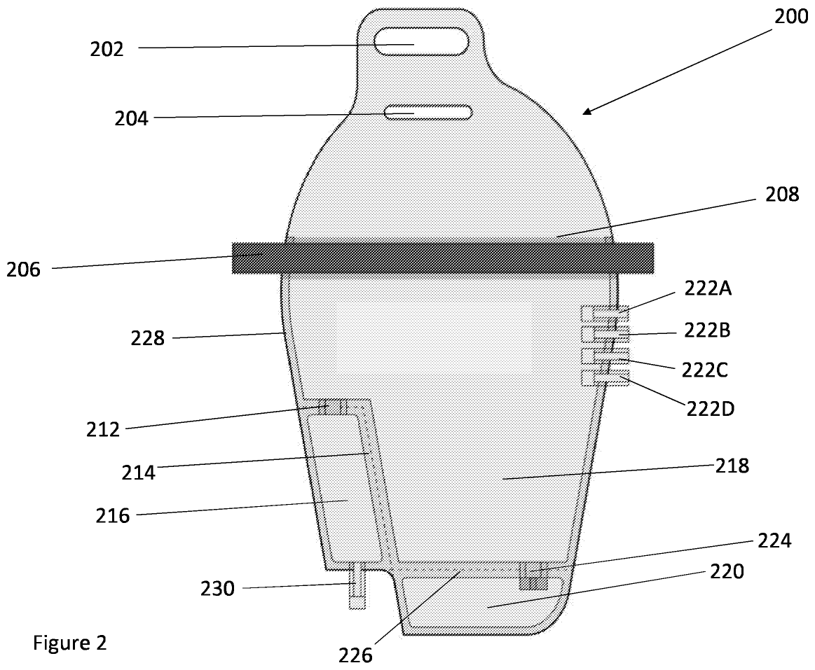

FIG. 2 shows a flexible faecal matter collection container 200 comprising a handle 202 and a clip aperture 204. In this embodiment the handle comprises a aperture in the flexible material of the container, though it could be formed of an additional loop of material. Similarly, the clip aperture 204 is formed of an aperture in the flexible container, though in other embodiments the clip aperture may be replaced by any attachment means including adhesive, a snap on connector, hook and loop tape, poppers, buttons, fast release mechanism, a zip tie.

The embodiment shown in FIG. 2 further comprises a clamping region 208 which is a region indicated by the material of the container being a different colour to that surrounding it. Alternatively, or in addition, the clamping region may be shown by a line, an arrow, written instructions, tactile features or the like. A clamp 206 is also shown in place on the clamp locator. After a deposit and when the clamp is in place the portion of the flexible faecal matter collection container comprising the clip aperture and the handle may be removed as superfluous and to ensure that the container is not reused. This may be eased by including a destructible portion atop the clamp, on the same side of the clamp as the handle.

In this embodiment four fluid ports 222A-D are shown connected to the first compartment, these can be used to admit fluids such as saline or to emit further samples or prepared donations into external containers or a recipient.

The first compartment comprises a mesh, which allows liquid to flow through it into the second compartment which will substantially separate liquid from stool. This liquid can then flow through the sealable port 224 that leads to the sump 220. The sump is connected to the first compartment by a destructible portion being a line of weakening having perforations suitable for tearing by a user. In alternative embodiments, the destructible portion is one of: a cutting line comprising material for destruction by cutting, a heating line comprising a material for destruction or weakening by heating or any known method. The donated stool in the first compartment 218 can be processed by urging liquid from the first compartment or by simply allowing it to drain by gravity. The sealable port 224 may be sealed when sufficient liquid has been removed and the destructible portion 226 may be utilised to remove the sump from the rest of the collection container. This allows the liquid in the sump to be tested or destroyed separately.

A further compartment 216 which may be used to hold a sample of the stool from the first compartment 218. Stool may be urged through the sealable port 212 from the first compartment and into the removable sample compartment 216. An inlet/outlet port 230 is also attached to the removable sample compartment, this can be used to remove any waste liquid which makes its way into the removable sample compartment or to allow liquids in for tests, or to emit, under urging a portion of the sample into or onto a separate vessel or slide. The sealable port 212 can be sealed after a portion of the donated stool has been urged into the removable sample portion. The destructible portion 214 may then be utilised to allow a user to remove the sample for testing and to allow the remaining donation in the first compartment to be delivered to a separate destination.

The seal 228 surrounds the flexible faecal matter collection container with the exception of the top such that an opening is formed at the top of the container which can be opened up such that said container may be fitted to a toilet frame.

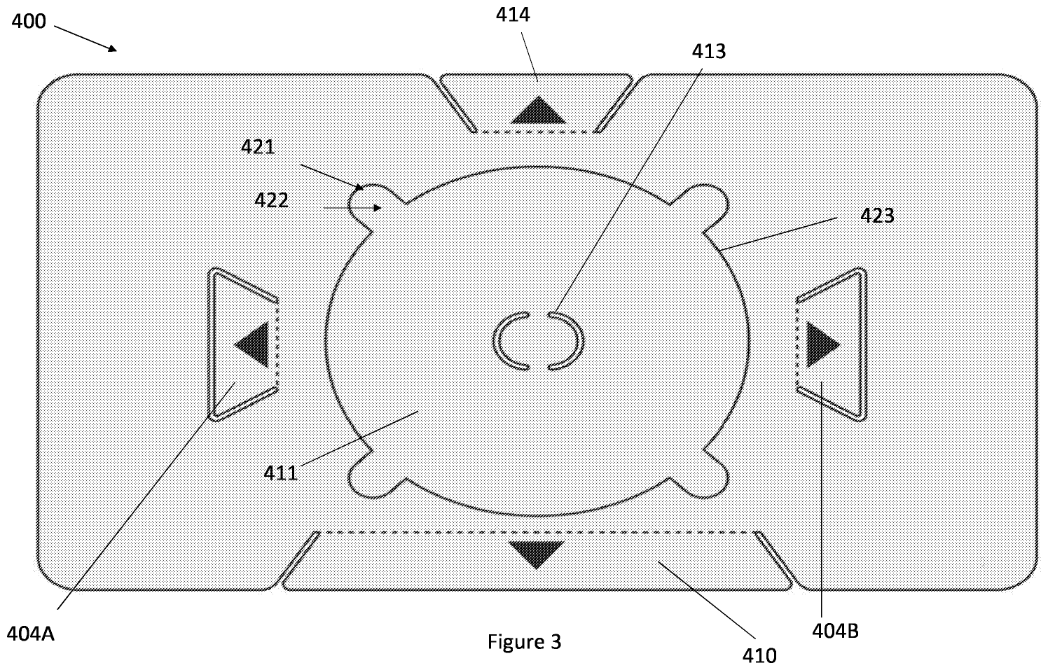

FIG. 3 shows a frame 400 for a toilet which may be fitted under a toilet seat. The embodiment shown comprises a urine shield 410 which comprises a deformable flap configured to be moved from a stowed position to a deployed position in which it is operable to deflect urine away from the opening. The embodiment shown further comprises a lid 411 which covers an opening. The lid 411 includes a handle 413 which is formed by bending pre-formed or pre-cut sections of the lid. The lid 411 is separable from the removing material of the frame 400 along a destructible line of weakening 423. The line of weakening 423 may be a partially cut through line in the material of the frame 400. The lid 411 is generally circular with four protrusions 422 extending radially outwardly from the circumference of the lid 411. The protrusions 422 initially align with notches 421 in the frame 400. Once the lid 411 is removed from the frame 400, it can be rotated before being replaced onto the frame. When replaced, the protrusions 422 no longer align with protrusions 421 and the lid 411 serves to close the opening in the frame and hide the deposited specimen from the donor or other person. The notches 422 prevent the detached lid 411 form falling onto the deposited specimen an causing contamination of that sample.

The embodiment shown further includes a flexible location tab 414 which can be in a stowed or deployed position such that when stowed several toilet frames may be stacked on top of each other. When in the deployed position the location tab is perpendicular to the surface of the frame which will abut the rim of the toilet. Different sizes of location tab may be used for different sizes of toilet such that when a flexible faecal matter collection container is fitted it fits into the toilet, preferably without touching the sides or the water in the bowl.

The toilet frame further comprises clips 404A and 404B which can be deployed or a stowed such that when stowed several toilet frames may be stacked on top of each other. When deployed the clips may be fitted with a with complimentary clip apertures on the specimen collection container. The clips may be replaced by any other connection means including adhesive, a snap on connector, hook and loop tape, poppers, buttons, fast release mechanism, zip ties.

The toilet frame may further include written instructions which detail how to fit the frame to the toilet and how to fit a specimen collection container to the frame. The instructions may be printed directly onto the surface of the frame. The instructions may also be in the form of an image of a deployed toilet frame.

The toilet frame may be formed of cardboard, a coated board material or a polymer.

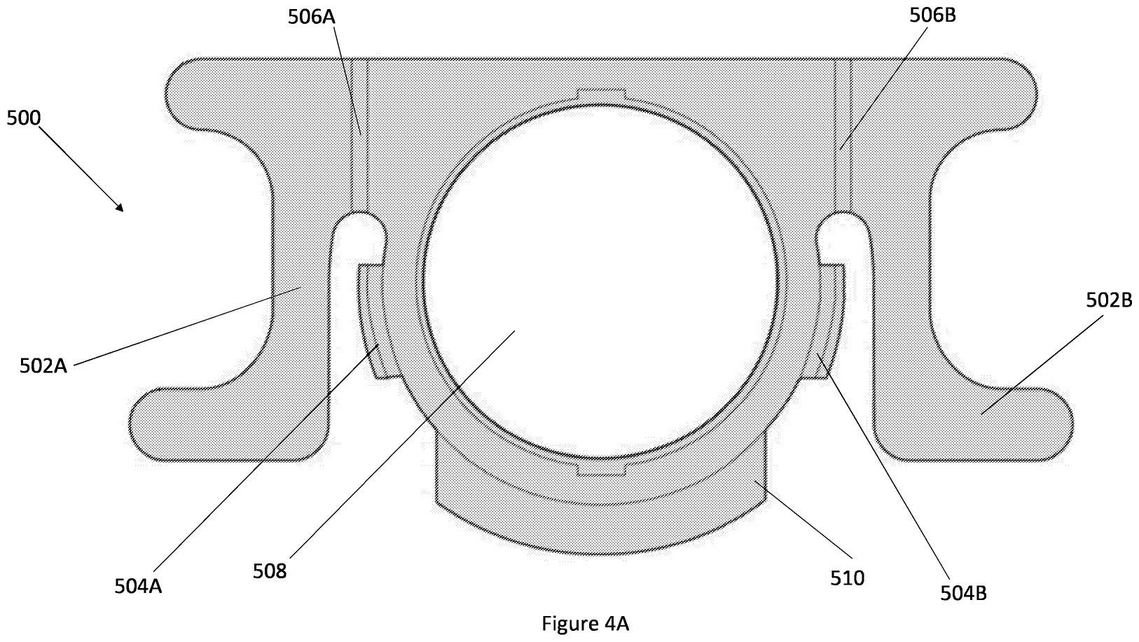

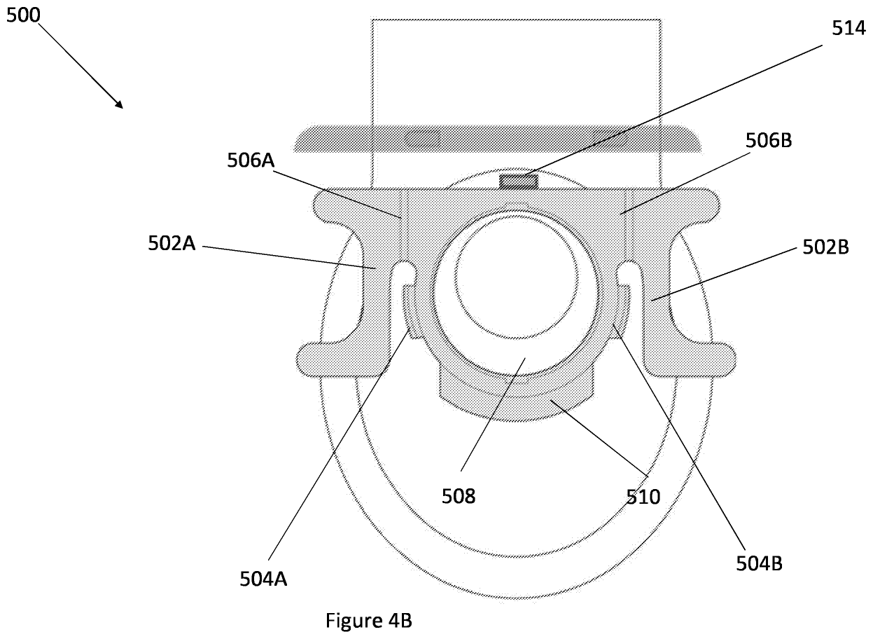

FIG. 4A shows a further embodiment of the toilet frame 500, comprising an aperture 508, support arms 502A, 502B attached to the frame by hinges 506A, 506B as well as a urine shield 510 comprising a flexible tab. The support arms can be retracted or deployed such that when retracted they have a smaller profile for transportation. The clips 504A and 504B are integrally formed with the frame.

FIG. 4B shows the toilet frame 500 of FIG. 4A further comprising a flexible location tab 514 which can be stowed or deployed. When deployed the location tab will facilitate correct fitting of the toilet frame and allow a user to place the frame in the correct position and orientation by making the location tab abut the rear of the toilet rim, ensuring that when the collection container is connected it is always in the optimal position.

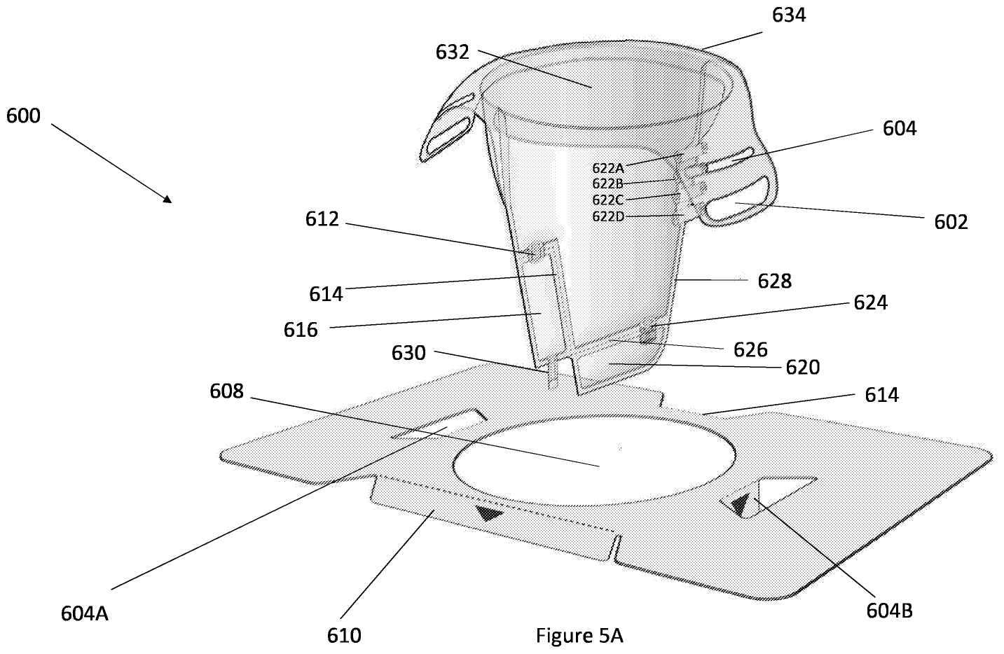

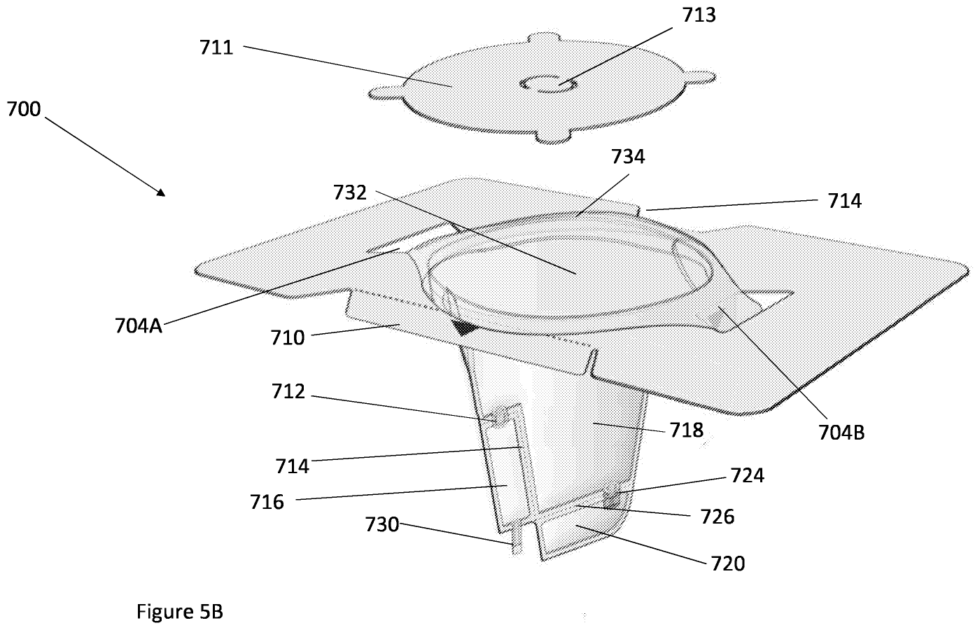

FIG. 5A shows a system 600 comprising the flexible faecal matter collection container of FIG. 2 and a deployed frame for a toilet as shown in FIG. 3. The flexible faecal matter collection container comprises an opening 632 and a rim 634.

The frame comprises a urine shield 610 which comprises a deformable flap configured to be moved from a stowed position to a deployed position in which it is operable to deflect urine away from the opening. The lid 711 (FIG. 5B) includes a handle 713 which is formed by bending pre formed or pre-cut sections of the lid. The lid 711 is separate from the frame to allow a collection container to be inserted into opening 608 and connected by clip apertures 604 to clips 604A and 604B.

A flexible location tab 614 which can be in a stowed or deployed position such that when stowed several toilet frames may be stacked on top of each other. When in the deployed position the location tab 614 is perpendicular to the surface of the frame which will abut the rim of the toilet.

The system of FIG. 5A also has a flexible faecal matter collection container comprising a handle 602 and a clip aperture 604. The collection container is mounted to the toilet support frame by placing the clips 604A and 604B through clip apertures 604.

Four fluid ports 622A-D connected to the first compartment can be used to admit fluids such as saline and glycerol to the first compartment 628 or to emit further samples or prepared donations into external containers or a recipient.

The first compartment 628 comprises a mesh (not shown), which allows liquid to flow through it into the second compartment which will substantially separate liquid from stool. This liquid can then flow through the sealable port 624 that leads to the sump 620. The sump 620 is connected to the first compartment by a destructible portion being a line of weakening 626 having perforations suitable for tearing by a user. In alternative embodiments, the destructible portion is one of: a cutting line comprising material for destruction by cutting, a heating line comprising a material for destruction or weakening by heating or any known method. The donated stool in the first compartment can be processed by urging liquid from the first compartment or by simply allowing it to drain by gravity. The sealable port 624 may be sealed when sufficient liquid has been removed and the destructible portion 626 may be utilised to remove the sump from the rest of the collection container. This allows the liquid in the sump to be tested or destroyed separately.

A further compartment 616 which may be used to hold a sample of the stool from the first compartment. Stool may be urged through the sealable port 612 from the first compartment and into the removable sample compartment 616. An inlet/outlet port 630 is also attached to the removable sample compartment, this can be used to remove any waste liquid which makes its way into the removable sample compartment or to allow liquids in for tests, or to emit, under urging a portion of the sample into or onto a separate vessel or slide. The sealable port 612 can be sealed after a portion of the donated stool has been urged into the removable sample portion. The destructible portion may then be utilised to allow a user to remove the sample for testing and to allow the remaining donation in the first compartment to be delivered to a separate destination.

FIG. 5B shows a system 700 comprising the flexible faecal matter collection container of FIG. 2 and a deployed frame for a toilet as shown in FIG. 3. The flexible faecal matter collection container comprises an opening 732 and a rim 734. The clip apertures 704 of the collection container are attached to the clips 704A and 704B of the frame.

The frame comprises a urine shield 710 which comprises a deformable flap configured to be moved from a stowed position to a deployed position in which it is operable to deflect urine away from the opening. The lid 711 includes a handle 713 which is formed by bending pre formed or pre-cut sections of the lid. The lid 711 is separate from the frame to allow a collection container to be inserted into opening 608 and connected by clip apertures 704 to clips 704A and 704B.

A flexible location tab 714 which can be in a stowed or deployed position such that when stowed several toilet frames may be stacked on top of each other. When in the deployed position the location tab 714 is perpendicular to the surface of the frame which will abut the rim of the toilet.

The system of FIG. 5B also has a flexible faecal matter collection container comprising a handle 702 and a clip aperture 704. The collection container is mounted to the toilet support frame by placing the clips 704A and 704B through clip apertures 604.

The first compartment 728 comprises a mesh (not shown), which allows liquid to flow through it into the second compartment which will substantially separate liquid from stool. This liquid can then flow through the sealable port 724 that leads to the sump 720. The sump 720 is connected to the first compartment by a destructible portion being a line of weakening 726 having perforations suitable for tearing by a user. In alternative embodiments, the destructible portion is one of: a cutting line comprising material for destruction by cutting, a heating line comprising a material for destruction or weakening by heating or any known method. The donated stool in the first compartment 718 can be processed by urging liquid from the first compartment or by simply allowing it to drain by gravity. The sealable port 724 may be sealed when sufficient liquid has been removed and the destructible portion 726 may be utilised to remove the sump from the rest of the collection container. This allows the liquid in the sump to be tested or destroyed separately.

A further compartment 716 which may be used to hold a sample of the stool from the first compartment. Stool may be urged through the sealable port 712 from the first compartment and into the removable sample compartment 716. An inlet/outlet port 730 is also attached to the removable sample compartment, this can be used to remove any waste liquid which makes its way into the removable sample compartment or to allow liquids in for tests, or to emit, under urging a portion of the sample into or onto a separate vessel or slide. The sealable port 712 can be sealed after a portion of the donated stool has been urged into the removable sample portion. The destructible portion may then be utilised to allow a user to remove the sample for testing and to allow the remaining donation in the first compartment to be delivered to a separate destination.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A specimen collection container for the collection and processing of faeces and configured for use with a toilet frame, the container comprising:
a sealable opening for receiving stool directly from a donor;
a first compartment for receiving and holding stool, said first compartment coupled to said sealable opening; and
one or more further compartments configured to receive a sample from the first compartment, wherein the further compartments comprise a further sealable opening.

2. The specimen collection container according to claim 1, further comprising at least one port connected to said first compartment and being configured to pass at least a portion of the stool.

3. The specimen collection container according to claim 45, wherein the first compartment and the second compartment are separated by a filter which allows fluid ingress to the second compartment from the first compartment.

4. The specimen collection container according to claim 3, wherein the filter comprises a porous membrane.

5. The specimen collection container according to claim 3, wherein the filter is a constriction in the material of the container between the first compartment and the second compartment.

6. The specimen collection container according to claim 45, the first compartment is tapered to the second compartment for collecting and holding fluid.

7. The specimen collection container according to claim 45, further comprising a sump in fluid communication with said second compartment for collecting and holding fluid.

8. The specimen collection container of claim 7, wherein the sump is in fluid communication with said second compartment for collecting and holding fluid via at least one of a one way valve, a drain hole and an orifice.

9. The specimen collection container according to claim 7, wherein an absorbent material is located in said sump.

10. The specimen collection container according to claim 7, wherein a device for detecting and indicating the pH of the fluid is located in said sump.

11. The specimen collection container according to claim 7, wherein said second compartment for collecting and holding fluid is fluidly isolated from said sump by a seal.

12. The specimen collection container according to claim 11, wherein the seal is at least one of the following; a welding line comprising material for welding, a clamp and clamping line comprising material suitable for clamping, an adhesive, magnetic fasteners, a hook and loop fastener, a zip-lock, and a snap fit seal.

13. The specimen collection container according to claim 7, wherein a destructible portion is located between said first compartment and sump to allow removal of said sump from said first compartment.

14. The specimen collection container according to claim 1, wherein the collection container is formed of a material suitable for visual inspection of the contents.

15. The specimen collection container according to claim 1, wherein the collection container is formed of a deformable material suitable for manipulation of the contents by hand.

16. The specimen collection container according to claim 44, wherein the second compartment being is detachable from the collection container.

17. The specimen collection container according to claim 16, wherein the sealable port is sealable by at least one of the following; welding section comprising material for welding including, a clamp and clamping line comprising material suitable for clamping, an adhesive, a zip-lock, and a snap fit seal.

18. The specimen collection container according to claim 16, wherein the sealable port has an internal diameter of up to 10 mm.

19. The specimen collection container according to claim 16, wherein a destructible portion is located between the second compartment for transferring stool and said first compartment to allow removal of the second compartment for transferring stool from said first compartment.

20. The specimen collection container according to claim 16, wherein the second compartment for transferring stool further comprises at least one sealable external port.

21. The specimen collection container according to claim 1, wherein the sealable opening is held closed by a deformable material clamp circumscribing the sealable opening until a force is applied to deform said deformable material clamp moving same to an open position.

22. The specimen collection container of claim 1, comprising an indicated clamp area which can be closed by a clamp for closing said sealable opening.

23. The specimen collection container according to claim 1, further comprising at least one attachment element for attaching the container to a toilet support frame.

24. The specimen collection container according to claim 23, wherein the at least one attachment element comprises an aperture in the material of the container wherein the aperture is configured to engage with a clip element on a support frame.

25. The specimen collection container according to claim 1, further comprising at least one handle.

26. The specimen collection container according to claim 1, further comprising a timing mechanism.

27. The specimen collection container according to claim 26, wherein the timing mechanism is a stopwatch.

28. The specimen collection container according to claim 27, wherein the stopwatch is configured to start recording time when the sealable opening is sealed.

29. The specimen collection container according to claim 1, wherein the specimen is faecal matter or an IMMP.

30. The specimen collection container according to claim 1, wherein the first compartment comprises a filter configured to separate the first compartment into two sub-compartments.

31. The specimen collection container as claimed in claim 31, wherein the first compartment comprises more than one filter configured to separate the first compartment into more than two compartments.

32. The specimen collection container as claimed in claim 31, wherein filters are arranged at least one of longitudinally and horizontally within the first compartment.

33. The specimen collection container as claimed in claim 31, wherein the filters are sequentially graduated filters.

34. A toilet support frame for use with a specimen collection container, the frame comprising:
an opening through which a donor may deposit a stool sample;
at least one clip for engaging an attachment element of a flexible faecal matter collection container;
a urine shield configured to deflect urine away from said opening.

35. A toilet support frame according to claim 34, wherein the urine shield comprises a deformable flap configured to be moved from a stowed position to a deployed position in which it is operable to deflect urine away from the opening.

36. A toilet support frame according to claim 34, comprising a location tab operable to engage with a toilet to secure the toilet support frame in the correct orientation and position relative to the toilet bowl.

37. The toilet support frame according to claim 34, further comprising a plurality of support arms for positioning under a toilet seat or directly on a toilet bowl.

38. The toilet support frame according to claim 37, wherein the plurality of support arms are attached to the frame by a hinge.

39. The toilet frame according to claim 38, wherein the hinge is a living hinge.

40. The toilet frame according to claim 34, wherein said frame is a unitary construction.

41. The toilet frame according to claim 34, wherein said frame has a fluid resistant surface.

42. The toilet frame according to claim 34, comprising a removable and replaceable lid configured to close the opening through which a donor may deposit a stool sample.

43. A system for the collection and processing of faeces comprising:
a specimen collection container comprising:
a sealable opening for receiving stool directly from a donor,
a first compartment for receiving and holding stool, said first compartment coupled to said sealable opening, and
one or more further compartments configured to receive a sample from the first compartment, wherein the further compartments comprise a further sealable opening; and
a toilet support frame comprising:
an opening through which a donor may deposit a stool sample,
at least one clip for engaging an attachment element of a flexible faecal matter collection container, and
a urine shield configured to deflect urine away from said opening.

44. The specimen collection container according to claim 1, wherein the one or more further compartments comprises a second compartment, and wherein the sealable opening is a sealable port configured to transfer stool from the first compartment to the second compartment.

45. The specimen collection container according to claim 1, wherein the one or more further compartments comprises a second compartment, wherein the second compartment is configured for collecting and holding fluid received from the donor through the sealable opening, and wherein the second compartment is in fluid communication with the first compartment and configured to substantially separate the fluid from the stool.
